(19) United States
(12) Patent Application Publication
(10) Pub. No.: US 2026/0188477 A1
(43) Pub. Date: Jul. 2, 2026

(54) STATE DETERMINATION APPARATUS, MAINTENANCE MANAGEMENT APPARATUS, INFORMATION PROCESSING APPARATUS, X-RAY CT APPARATUS, AND PROGRAM

(71) Applicant: FUJIFILM Corporation, Tokyo (JP)

(72) Inventor: Kazunari HONDA, Tokyo (JP)

(73) Assignee: FUJIFILM Corporation, Tokyo (JP)

(21) Appl. No.: 19/424,187

(22) Filed: Dec. 18, 2025

(30) Foreign Application Priority Data
Dec. 26, 2024 (JP) 2024-230904

(51) Int. Cl. G16H 40/40; G16H 30/20

(52) U.S. Cl. CPC G16H 40/40; G16H 30/20

(57) ABSTRACT

Provided are a state determination apparatus, a maintenance management apparatus, an information processing apparatus, and an X-ray CT apparatus that can appropriately determine a state of a slip ring. The state determination apparatus includes a lighting device that irradiates a slip ring with light, a camera that captures an image of a region including the slip ring, and a processor that recognizes the image acquired via the camera and determines a state of an object based on a recognition result of the image. The state of the object may include at least one of a degree of contamination of the slip ring, a degree of contamination of an inter-ring region of the slip ring, or an amount of deposits in the inter-ring region. The processor calculates a feature value from the image, and determines whether maintenance is necessary based on the feature value and a threshold value that is set.

### CROSS-REFERENCE TO RELATED APPLICATION

The present application claims priority under 35 U.S.C § 119(a) to Japanese Patent Application No. 2024-230904 filed on Dec. 26, 2024, which is hereby expressly incorporated by reference, in its entirety, into the present application.

### BACKGROUND OF THE INVENTION

### 1. Field of the Invention

The present disclosure relates to a state determination apparatus, a maintenance management apparatus, an information processing apparatus, an X-ray CT apparatus, and a program, and particularly relates to a technology of determining a surface state of a slip ring used in a rotary sliding power-feeding mechanism.

### 2. Description of the Related Art

JP2014-89100A discloses a technology related to an apparatus and a method for diagnosing a sliding state of a rotary electric machine, in which abnormal sliding of the rotary electric machine can be detected at an early stage, and a stop period and a maintenance cost of the rotary electric machine can be reduced. The apparatus for diagnosing a sliding state of a rotary electric machine disclosed in JP2014-89100A includes a light source that emits light incident on a sliding surface between a current collecting brush and a rotating body surface of the rotary electric machine, a light-receiving unit that receives reflected light from the sliding surface, and a determination unit that processes a signal from the light-receiving unit, in which the determination unit detects an increase in a specific wavelength component of the reflected light to determine an abnormality of the sliding state on the rotating body surface of the rotary electric machine.

### SUMMARY OF THE INVENTION

The technology disclosed in JP2014-89100A has the following issues.
[Issue 1] The technology disclosed in JP 2014-89100A is configured to detect (estimate) the extent of deposits on the sliding surface by detecting the increase in the specific wavelength component of a spectral spectrum of the reflected light, and cannot detect irregularities such as scratches on the sliding surface.
[Issue 2] A change in appearance of the sliding surface cannot be visually checked.
[Issue 3] A threshold value to be compared in the time of measurement is determined, so that a result of the abnormality determination varies even in a case in which intensities of the reflected light are the same. Therefore, there is a risk that the rotary electric machine may be used in an abnormal wear state.
[Issue 4] Since only the specific wavelength component is used for the detection, it is difficult to determine a variation in dirt on the same ring of the slip ring, a variation in dirt of a plurality of rings, a dirty state in a groove between the rings, and the like.
[Issue 5] For a plurality of types of rings such as a power slip ring and a control slip ring, even in a case in which the extent of dirt is the same, a tolerance differs depending on a type of the ring, so that it is not appropriate to perform the abnormality determination by uniform standards.

The present disclosure has been made in view of such circumstances, and an object thereof is to provide a state determination apparatus, a maintenance management apparatus, an information processing apparatus, an X-ray CT apparatus, and a program that can appropriately determine a state of a slip ring by eliminating at least some of the plurality of issues described above.

A first aspect of the present disclosure relates to a state determination apparatus comprising: a lighting device that irradiates a slip ring with light; a camera that captures an image of a region including the slip ring; and a processor that recognizes the image acquired via the camera and determines a state of an object based on a recognition result of the image.

According to the first aspect, the lighting device irradiates the slip ring with light, and the image of the region including the slip ring is captured by the camera. The processor can recognize the object in the image from the image captured by the camera and determine the state of the object. Examples of the object include the slip ring, the inter-ring region, and the deposits. The inter-ring region is a non-slip ring region adjacent to the slip ring, and is typically a region of the groove between the rings. It is possible to quantitatively evaluate dirt of the object, scratches (irregularities) on the surface, the amount of deposits, and the like by using the image obtained from the camera. Further, it is also possible to visually check the state of the object by displaying the image. The term “ring” is not limited to shapes including an arc, and includes the concept of ring-like shapes, and also includes shapes with discontinuous portions such as C-rings.

A second aspect relates to the state determination apparatus according to the first aspect, in which the state of the object may include at least one of a degree of contamination of the slip ring, a degree of contamination of an inter-ring region of the slip ring, or an amount of deposits in the inter-ring region. The term “contamination” includes the concept of dirt, scratches, or a combination thereof.

A third aspect relates to the state determination apparatus according to the first or second aspect, in which the processor may calculate a feature value from the image, and determine whether maintenance is necessary based on the feature value and a threshold value that is set.

The state of the object can be quantitatively evaluated by using the feature value calculated from the image. According to the third aspect, the degree of contamination that requires maintenance can be determined by comparing with the threshold value.

A fourth aspect relates to the state determination apparatus according to the third aspect, that may further comprise: a storage device that stores at least one of the image, the feature value calculated from the image, the threshold value, or a determination result of the state.

A fifth aspect relates to the state determination apparatus according to the fourth aspect, in which the processor may notify an external apparatus of information stored in the storage device via a network.

A sixth aspect relates to the state determination apparatus according to any one of the third to fifth aspects, in which the threshold value may be set differently depending on a type of the slip ring.

For example, the threshold value of the degree of contamination that requires maintenance may be different between the power slip ring and the control slip ring.

A seventh aspect relates to the state determination apparatus according to any one of the first to sixth aspects, in which the image may include at least one of a power slip ring for power transmission, or a control slip ring for signal transmission.

An eighth aspect relates to the state determination apparatus according to any one of the first to seventh aspects, in which the image may include a groove of an inter-ring region of the slip ring.

A ninth aspect relates to the state determination apparatus according to any one of the first to eighth aspects, in which a color of an inter-ring region of the slip ring may be a color other than black.

A tenth aspect relates to the state determination apparatus according to any one of the first to ninth aspects, in which the image may be captured using diffused light.

An eleventh aspect relates to the state determination apparatus according to the tenth aspect, in which the lighting device may irradiate the region including the slip ring with light from an oblique direction.

A twelfth aspect relates to the state determination apparatus according to the tenth or eleventh aspect, in which a plurality of the lighting devices that irradiate the region including the slip ring with light from a plurality of directions may be provided.

A thirteenth aspect relates to the state determination apparatus according to any one of the tenth to twelfth aspects, that may further comprise: a light-shielding guard that suppresses direct incidence of light from the lighting device to the camera.

A fourteenth aspect relates to the state determination apparatus according to any one of the first to ninth aspects, in which the image may be captured using specularly reflected light.

A fifteenth aspect relates to the state determination apparatus according to the fourteenth aspect, that may further comprise: a half mirror that is disposed between the lighting device and the slip ring and reflects the specularly reflected light from the slip ring toward the camera.

A sixteenth aspect relates to the state determination apparatus according to any one of the first to fifteenth aspects, in which the processor may issue a warning in a case in which an abnormality of the object is detected.

A seventeenth aspect relates to the state determination apparatus according to any one of the first to sixteenth aspects, that may further comprise: a display device that displays a determination result of the state.

An eighteenth aspect relates to the state determination apparatus according to any one of the first to seventeenth aspects, in which the slip ring may be provided on a rotating member that rotates around a subject in a medical imaging apparatus.

A nineteenth aspect relates to the state determination apparatus according to any one of the first to eighteenth aspects, in which the processor may determine an acquisition frequency of the image based on a feature value calculated from the image.

A twentieth aspect relates to the state determination apparatus according to the nineteenth aspect, in which the processor may set a condition for changing the acquisition frequency of the image, and change the acquisition frequency of the image in a case in which the feature value satisfies the condition.

A twenty-first aspect relates to the state determination apparatus according to the nineteenth or twentieth aspect, in which the processor may change the acquisition frequency of the image in accordance with a difference between the feature value and a threshold value that is set.

A twenty-second aspect relates to the state determination apparatus according to the twentieth aspect, in which the processor may acquire condition setting information that defines the condition via a network, and change the condition based on the condition setting information.

A twenty-third aspect relates to a maintenance management apparatus connected to a system including the state determination apparatus according to any one of the first to twenty-second aspects via a network, the maintenance management apparatus comprising: a second processor different from a first processor that is the processor included in each of a plurality of the systems, in which the second processor acquires information from the plurality of systems via the network, determines setting information applied to the determination in the system based on the information acquired from the plurality of systems, and provides the setting information to the systems via the network.

A twenty-fourth aspect relates to the maintenance management apparatus according to the twenty-third aspect, in which the information acquired by the second processor from the plurality of systems may include at least one of a feature value calculated from the image of the system, a status of the system, or operation information of the system, and the setting information may include at least one of a threshold value used for the determination, a type of the feature value used for the determination, an acquisition frequency of the image, or a condition for changing the acquisition frequency of the image.

A twenty-fifth aspect relates to the maintenance management apparatus according to the twenty-fourth aspect, in which the second processor may acquire the feature value and the status indicating whether a system outage has occurred from the plurality of systems, and determine at least one of the threshold value used for the determination or the type of the feature value used for the determination based on a dataset including the acquired feature value and the acquired status.

A twenty-sixth aspect relates to the maintenance management apparatus according to the twenty-fourth or twenty-fifth aspect, in which the operation information of the system may include information on the number of revolutions and a rotational speed of a rotating member provided with the slip ring in the system, and the second processor may acquire the information on the number of revolutions and the rotational speed of each system and the feature value from the plurality of systems, classify the plurality of systems using the information on the number of revolutions and the rotational speed, calculate a representative value of an amount of change in the feature value for each class of the classified systems, and determine the condition for changing the acquisition frequency of the image for each class based on a comparison result between the representative value and an estimated value of the amount of change as a reference.

A twenty-seventh aspect relates to an X-ray CT apparatus comprising: an X-ray source that irradiates a subject with X-rays; an X-ray detector that is disposed to face the X-ray source and detects the X-rays transmitted through the subject; a rotating member that is provided with the X-ray source and the X-ray detector and rotates around the subject; a slip ring that is provided on the rotating member; a lighting device that irradiates the slip ring with light; a camera that captures an image of a region including the slip ring; and a processor that recognizes the image acquired via the camera and determines a state of an object based on a recognition result of the image.

The X-ray CT apparatus according to the twenty-seventh aspect may have a configuration including the same specific aspects as the state determination apparatus according to any one of the second to twenty-second aspects.

A twenty-eighth aspect relates to an information processing apparatus comprising: a processor, in which the processor acquires an image of a region including a slip ring, recognizes the image, and determines a state of an object based on a recognition result of the image.

The information processing apparatus according to the twenty-eighth aspect may have a configuration including the same specific aspects as the state determination apparatus according to any one of the second to twenty-second aspects.

A twenty-ninth aspect relates to a program causing a computer to implement: a function of acquiring an image of a region including a slip ring; a function of recognizing the image; and a function of determining a state of an object based on a recognition result of the image.

The program according to the twenty-ninth aspect may have a configuration including the same specific aspects as the state determination apparatus according to any one of the second to twenty-second aspects. The present disclosure also encompasses a tangible, non-transitory computer-readable storage medium on which the program according to the twenty-ninth aspect is stored.

According to the present disclosure, it is possible to appropriately determine the state of the object in the region including the slip ring based on the recognition result of the image of the region including the slip ring.

### BRIEF DESCRIPTION OF THE DRAWINGS

FIG. 1 is a diagram illustrating a basic configuration of a system including an X-ray CT apparatus and a maintenance management apparatus according to an embodiment of the present disclosure.

FIG. 2 is a diagram illustrating an overview of a state determination apparatus implemented in the X-ray CT apparatus 1.

FIG. 3 is an example of an image obtained by imaging a region including a slip ring.

FIG. 4 is a schematic diagram illustrating a disposition example 1 of a lighting device and a camera.

FIG. 5 is a schematic diagram illustrating a disposition example 2 of the lighting device and the camera.

FIG. 6 is a block diagram illustrating a hardware configuration example of an information processing apparatus used in an operator console.

FIG. 7 is a graph illustrating a transition of a measured value of a certain feature value.

FIG. 8 is a graph illustrating an example of a frequency change condition set in advance.

FIG. 9 is a flowchart illustrating an example of a process of changing a measurement frequency of the feature value.

FIG. 10 is a diagram illustrating an example of a configuration in which a plurality of CT systems are connected to the maintenance management apparatus via a network.

FIG. 11 is a graph illustrating an example of a default threshold value setting.

FIG. 12 is a graph including data in a case in which desired classification cannot be performed with the default threshold value.

FIG. 13 is a graph illustrating an example of a case in which a new threshold value is set again using a dataset of FIG. 12.

FIG. 14 is a graph illustrating an example of a case in which a new threshold value is set by changing the feature value used for the classification.

FIG. 15 is a conceptual diagram of the classification of the plurality of systems.

FIG. 16 is a graph illustrating an average of amounts of change in the feature value for each class.

FIG. 17 is an example of a measurement frequency function.

### DESCRIPTION OF THE PREFERRED EMBODIMENTS

Hereinafter, detailed description of preferred embodiments of the present invention will be made with reference to the accompanying drawings. In the present specification, the same reference numeral will be given to the same configuration element, and duplicate description thereof will be omitted as appropriate.

### Configuration Example of System

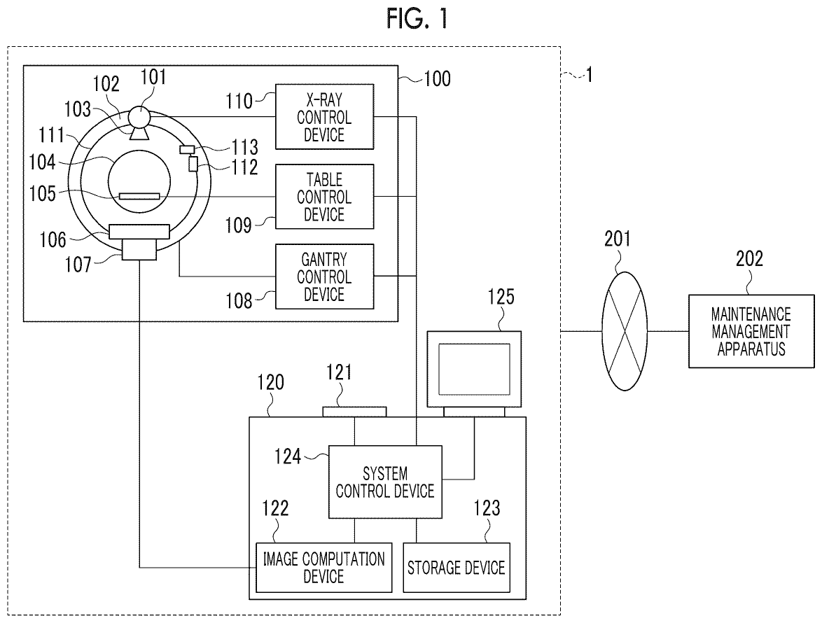

FIG. 1 is a diagram illustrating a basic configuration of a system including an X-ray CT apparatus 1 and a maintenance management apparatus 202 according to the embodiment of the present disclosure. The X-ray CT apparatus 1 comprises a scan gantry unit 100 and an operator console 120.

The scan gantry unit 100 comprises an X-ray tube 101, a rotating disk 102, a collimator 103, an X-ray detector 106, a data collection device 107, a table 105, a gantry control device 108, a table control device 109, and an X-ray control device 110.

The X-ray tube 101 and a high-voltage generation unit (not illustrated) are mounted on the rotating disk 102, and rotate together with the rotating disk 102. The other components are not mounted on the rotating disk 102 and are stationary. The rotating disk 102 comprises a slip ring 111. The slip ring 111 electrically connects the components mounted on the rotating disk 102 to the stationary components. The high-voltage generation unit is connected to a rotating side of the slip ring 111, and a DC high current conversion unit is connected to a stationary side of the slip ring 111. As a result, the DC high current conversion unit and the high-voltage generation unit are electrically connected via the slip ring 111. The slip ring 111 includes a power slip ring for power transmission and a control slip ring for signal transmission such as control signal transmission.

In addition, the X-ray CT apparatus 1 comprises a lighting device 112 that irradiates a surface of the slip ring 111 with light and a camera 113 that captures an image of a region including the slip ring 111 irradiated with light by the lighting device 112, as means for diagnosing a state of the slip ring 111.

The collimator 103 controls an irradiation range of X-rays emitted from the X-ray tube 101. The X-ray detector 106 is disposed to face the X-ray tube 101, and detects the X-rays transmitted through a subject. The rotating disk 102 comprises an opening part 104 through which the subject mounted on the table 105 enters. The rotating disk 102 comprises the X-ray tube 101 and the X-ray detector 106, and a drive unit (not illustrated) that rotates around the subject. The X-ray detector 106 has a configuration in which a plurality of detection elements are disposed in the rotation direction of the rotating disk 102. In a case in which the plurality of detection elements are arranged in one row in the rotation direction, the plurality of detection elements may be arranged in a plurality of rows (for example, 64 rows) in a direction of the rotation axis of the rotating disk 102. The rotation direction of the rotating disk 102 will be also referred to as a “channel direction”. The direction of the rotation axis of the rotating disk 102 will be also referred to as a “slice direction”.

The X-ray control device 110 includes an X-ray high-voltage device and controls power supplied to the X-ray tube 101. The data collection device 107 is a device that converts the X-rays detected by the X-ray detector 106 into a predetermined electrical signal. The gantry control device 108 is a device that controls the rotation of the rotating disk 102. The table control device 109 is a device that controls up-down movement and front-rear movement of the table 105. The front-rear movement of the table 105 means movement in the direction of the rotation axis of the rotating disk 102.

The operator console 120 comprises an input device 121, an image computation device 122, a storage device 123, a system control device 124, and a display device 125. The input device 121 is a device for inputting a subject name, an examination date and time, imaging conditions, and the like, and is specifically a keyboard, a pointing device, or the like. The image computation device 122 is a device that reconstructs a CT image by performing computation processing on measurement data sent from the data collection device 107, and is specifically a central processing unit (CPU) that executes the computation processing, a dedicated computing circuit, or a combination thereof.

The display device 125 is a device that displays the CT image or the like created by the image computation device 122. The storage device 123 is a device that stores data collected by the data collection device 107 and data such as the CT image created by the image computation device 122. The system control device 124 is a device that controls these devices, the gantry control device 108, the table control device 109, and the X-ray control device 110.

The X-ray control device 110 supplies a tube current and a tube voltage that are controlled such that the imaging conditions (tube voltage and the like) input from the input device 121 are satisfied to the X-ray tube 101. The X-ray tube 101 is an example of an “X-ray source” according to the present disclosure.

The X-rays emitted from the X-ray tube 101 and transmitted through the subject are detected by the X-ray detection element provided in the X-ray detector 106. During this period, the rotating disk 102 rotates the X-ray tube 101 and the X-ray detector 106 to irradiate the subject from each direction with the X-rays and detect the X-rays. The gantry control device 108 controls the rotational speed of the rotating disk 102 such that the imaging conditions (scan speed and the like) input from the input device 121 are satisfied. In addition, during a period in which the X-rays are emitted and detected, the table 105 moves the subject in a body axis direction under the control of the table control device 109 to operate such that the imaging conditions (helical pitch and the like) input from the input device 121 are satisfied.

The output signal of the X-ray detector 106 is collected as projection data by the data collection device 107. The projection data collected by the data collection device 107 is transmitted to the image computation device 122. The image computation device 122 performs reconstruction computation on the projection data to generate the CT image. The reconstructed CT image is displayed on the display device 125 and is also stored in the storage device 123 as image data together with the imaging conditions.

The image computation device 122 further has a function as an image processing device that analyzes various feature values from the image captured by the camera 113.

The maintenance management apparatus 202 is a device that collects and manages information on the maintenance of the X-ray CT apparatus 1. The maintenance management apparatus 202 may be configured by one or a plurality of computers. The maintenance management apparatus 202 may be implemented by cloud computing.

The X-ray CT apparatus 1 and the maintenance management apparatus 202 are connected to a network 201. The network 201 may be a local area network, a wide area network, or a combination thereof.

In FIG. 1, one X-ray CT apparatus 1 is illustrated, but a plurality of X-ray CT apparatuses may be communicably connected to the maintenance management apparatus 202 via the network 201.

The X-ray CT apparatus 1 is an example of a “medical imaging apparatus” according to the present disclosure, and the rotating disk 102 is an example of a “rotating member” according to the present disclosure. The maintenance management apparatus 202 is an example of an “external apparatus” according to the present disclosure.

### Overview of State Determination Apparatus According to First Embodiment

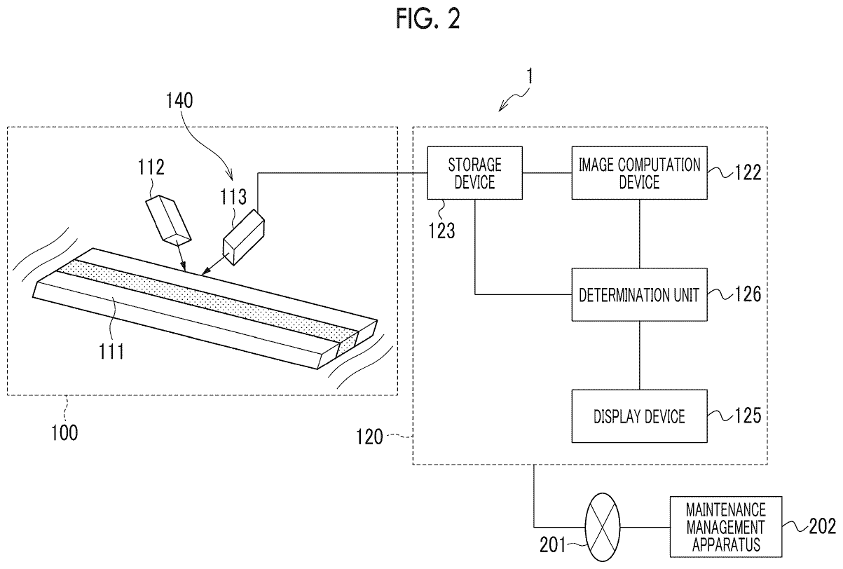

FIG. 2 is a diagram illustrating an overview of the state determination apparatus 140 implemented in the X-ray CT apparatus 1. The state determination apparatus 140 includes the lighting device 112, the camera 113, and the operator console 120. The lighting device 112 and the camera 113 are installed in the vicinity of the slip ring 111. The camera 113 includes an optical system, an image sensor, and a signal processing circuit (none of which are illustrated). The optical system includes one or more lenses such as a focus lens. The image sensor may be, for example, a charge coupled device (CCD) image sensor or a complementary metal-oxide semiconductor (CMOS) image sensor. The camera 113 generates digital image data of an imaging target by processing the signal obtained from the image sensor by the signal processing circuit. The term “image” includes the concept of “image data”.

The operator console 120 functions as an information processing apparatus that controls the operation of the lighting device 112 and the camera 113 and analyzes the image obtained from the camera 113. The operator console 120 comprises the storage device 123, the image computation device 122, a determination unit 126, and the display device 125. The determination unit 126 may be implemented in the system control device 124.

The image captured by the camera 113 under any conditions is processed by the image computation device 122. The image computation device 122 recognizes the image acquired via the camera 113 and analyzes various feature values from the image. The determination unit 126 compares an amount of change in the feature value with a preset threshold value to determine the state of the surface of the slip ring 111. The measured value of the feature value calculated from the image is understood as a value reflecting the amount of change in the feature value.

The term “determination” includes the concept of discrimination, determination, and diagnosis. The determination unit 126 may determine not only the surface of the slip ring 111 but also a surface state of an inter-ring region of the slip ring 111. The threshold value is set, for example, as a criterion for abnormality determination of whether the degree of contamination (contamination degree) requires maintenance.

In a case in which the determination unit 126 determines that there is an abnormality on the surface of the slip ring 111 or the surface of the inter-ring region, a warning is issued on a screen of the display device 125. Further, the image captured by the camera 113 can be displayed on the display device 125. The image captured by the camera 113, the threshold value, the comparison result between the feature value and the threshold value, and the warning content are stored in the storage device 123 in association with each other.

The system of the X-ray CT apparatus 1 is connected to the network 201, and the maintenance management apparatus 202 is notified of a status of the system via the network 201.

The status of the system includes various parameters related to the state of the X-ray CT apparatus 1. The status of the system may include information such as a lifetime, a remaining usable period, and a maintenance timing of a target component, which are predicted based on the determination result of the determination unit 126, in addition to the determination result of the determination unit 126. For example, the maintenance management apparatus 202 may acquire information such as the number of days remaining until a tolerance margin is eliminated, regarding the contamination state of the surface of the slip ring 111, via the network 201.

In order to improve the determination accuracy in the determination unit 126, it is preferable that the image is captured at a plurality of locations of the slip ring 111, and it is also preferable that the image is captured a plurality of times.

It is desirable that a plurality of cameras 113 are installed, the position of the camera 113 is moved, the slip ring 111 is rotated for imaging, or the like, and the imaging is performed at a plurality of locations a plurality of times to acquire a plurality of images.

Alternatively, in order to improve the determination accuracy, the image computation device 122 performs various types of correction on the image to be compared. The correction processing includes, for example, image averaging and contrast conversion.

### Image Example

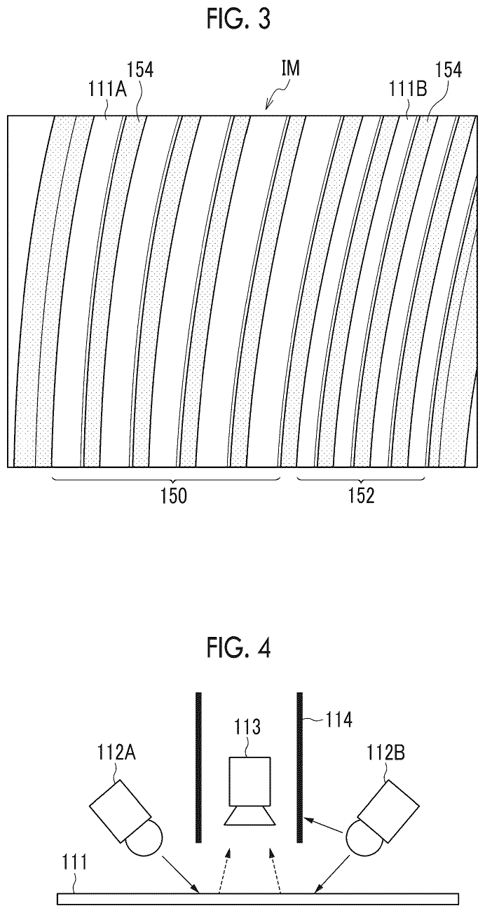

FIG. 3 is an example of an image IM obtained by imaging the region including the slip ring 111. The camera 113 can acquire the image IM of a region including a power slip ring 111A, a control slip ring 111B, and a groove 154 that is the inter-ring region. The power slip ring 111A functions as a contact point of power. The control slip ring 111B functions as a contact point of a control signal or the like. The respective rings of a plurality of power slip rings 111A arranged in a power slip ring unit 150 and a plurality of control slip rings 111B arranged in a control slip ring unit 152 may be or may not be arranged at equal intervals.

The power slip ring 111A is an example of a “power slip ring” according to the present disclosure, and the control slip ring 111B is an example of a “control slip ring” according to the present disclosure.

FIG. 3 illustrates an example of a clean state in which there is no dirt, scratches, or the like on the surfaces of the slip rings 111A and 111B, but shaving chips of a sliding member adhere to a sliding portion or a lubricating oil on the ring surface is soiled depending on the type of the slip ring due to the use of the X-ray CT apparatus 1. The control slip ring 111B is more susceptible to dirt than the power slip ring 111A. In the control slip ring 111B, an error may occur in communication or control due to dirt and the like.

A distribution of dirt adhesion is uneven even in the same ring. For the respective rings of the plurality of slip rings 111A and 111B, the manner of dirt differs among rings at different positions.

The groove 154 between the rings is short-circuited due to the accumulation of the shaving chips of the sliding member or the like. Examples of the sliding member include carbon, copper, and a silver alloy.

It is preferable that a gap (groove 154 between the rings) of the ring is a different color from the sliding member. For example, in a case in which the sliding member is carbon, the carbon shaving chips accumulated between the rings are black. Therefore, in order to accurately detect an adhesion state of the shaving chips between the rings by image recognition, the color of the gap between the rings is a color that is easily visually distinguishable from the color of the shaving chips, for example, green.

The slip ring 111 may be disposed in a form in which the plurality of rings are disposed concentrically with respect to a bore of the scan gantry unit 100, or may be disposed in a form in which the plurality of rings are disposed in a row in a Z-axis direction inside the bore.

### Disposition Example 1 of Lighting Device and Camera

FIG. 4 is a schematic diagram illustrating a disposition example 1 of lighting devices 112A and 112B and the camera 113. FIG. 4 is an example in a case in which the image is acquired using diffused light. A plurality of lighting devices 112A and 112B may be disposed to irradiate the region including the slip ring 111 with light from a plurality of directions. For example, as illustrated in FIG. 4, the lighting devices 112A and 112B are disposed to irradiate the slip ring 111 with light from an oblique direction. The camera 113 is disposed to image the slip ring 111 from a direction perpendicular to the slip ring 111.

Both the lighting devices 112A and 112B can be turned on (lit) at the same time, and only one of the lighting device 112A or the lighting device 112B can be turned on. Since the reflection state of the light changes by performing the imaging in a state in which only one of the lighting device 112A or the lighting device 112B is turned on, it is easy to detect the irregularities (scratches or dirt) of the slip ring 111.

A light-shielding guard 114 that suppresses direct incidence of the illumination light from the lighting devices 112A and 112B to the camera 113 may be provided around the camera 113. One or more cameras 113 may be used. The number of lighting devices 112A and 112B is determined in accordance with the number of cameras 113.

### Disposition Example 2 of Lighting Device and Camera

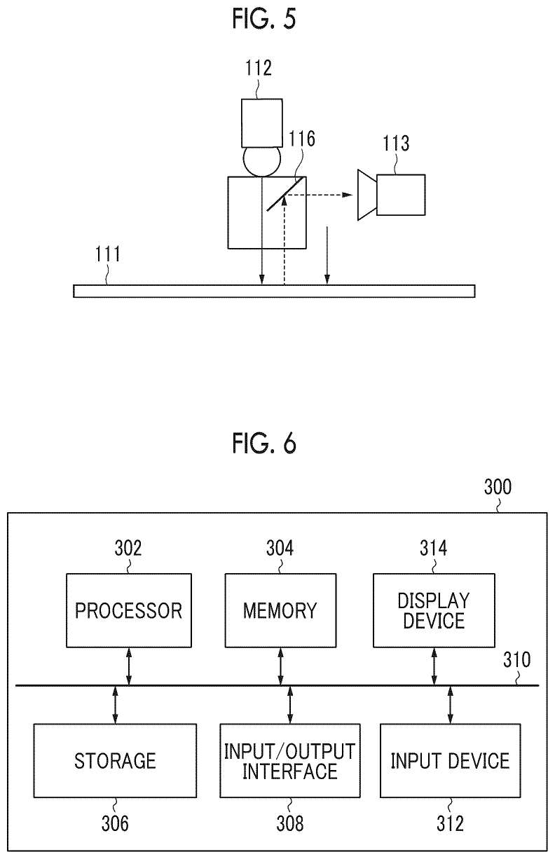

FIG. 5 is a schematic diagram illustrating a disposition example 2 of the lighting device 112 and the camera 113. FIG. 5 is an example in a case in which the image is acquired using specularly reflected light.

The lighting device 112 is disposed to irradiate the slip ring 111 with light from a direction perpendicular to the slip ring 111. The camera 113 is disposed in parallel to the slip ring 111. A half mirror 116 is disposed between the lighting device 112 and the camera 113, and is configured to reflect the specularly reflected light from the slip ring 111 by the half mirror 116 and direct the specularly reflected light to the camera 113. A light emitting diode (LED) can be used as the lighting device 112. By using the LED, the lighting device 112 can be disposed in a small space. With the configuration of FIG. 5, the camera 113 can be disposed in a small space by bending the specularly reflected light by 90 degrees by the half mirror 116.

Since the slip ring 111 is metal, the specularly reflected light is strong in a case in which the surface state is clean. In a case in which the slip ring 111 becomes dirty, the specularly reflected light is weakened, so that the difference in the surface state can be detected.

### Example of Hardware Configuration of Information Processing Apparatus

FIG. 6 is a block diagram illustrating a hardware configuration example of an information processing apparatus 300 used in the state determination apparatus 140. The information processing apparatus 300 may be a personal computer, a workstation, or a server computer. The information processing apparatus 300 comprises a processor 302, a memory 304 that is a main storage device, a storage 306 that is an auxiliary storage device, an input/output interface 308, and a bus 310.

The processor 302 includes a central processing unit (CPU). The processor 302 may include a graphics processing unit (GPU). In addition, the processor 302 may include hardware of a digital signal processor (DSP), an application-specific integrated circuit (ASIC), a programmable logic device (PLD), and the like.

The processor 302 is connected to the memory 304, the storage 306, the input/output interface 308, the input device 312, and the display device 314 via the bus 310.

The memory 304 includes a random access memory (RAM). The memory 304 may include a read only memory (ROM). The storage 306 may be, for example, a hard disk drive (HDD), a solid-state drive (SSD), or a plurality of combinations thereof. In addition, the storage 306 may include an external storage device such as a removable medium.

The storage device including the memory 304 and the storage 306 stores programs, data, or the like for implementing various functions of the information processing apparatus 300. The processor 302 executes the programs stored in the memory 304 to implement various functions. The processor 302 controls each unit of the information processing apparatus 300 and various devices and units provided in the X-ray CT apparatus 1, and performs various types of processing.

The input/output interface 308 includes a communication interface connectable to an electric communication line such as a local area network, a connection interface connectable to an external apparatus, and the like. As the connection interface connectable to the external apparatus, for example, a Universal Serial Bus, a High-Definition Multimedia Interface (HDMI) (HDMI is a registered trademark), and the like can be used.

The processor 302 communicates with various devices of the X-ray CT apparatus 1 and/or the external apparatus via the input/output interface 308 to transmit and receive necessary information.

The input device 312 includes, for example, a keyboard, a pointing device such as a mouse, a numeric keypad, various switch buttons, and the like. The input device 312 may include an audio input device. Additionally, the input device 312 may be a touch panel-type input device that is configured integrally with a display screen of the display device 314.

The display device 314 is configured by, for example, a liquid crystal display, an organic electroluminescence (OEL) display, a projector, or an appropriate combination thereof. The display device 314 displays various types of information in addition to the image captured by the X-ray CT apparatus 1. The display device 314 is used as a part of the user interface in a case in which an input from the input device 312 is received. The display device 314 is not limited to a single display device and can also be in a multi-display form comprising a plurality of display devices. The input device 312 and the display device 314 are used as the input device 121 and the display device 125 illustrated in FIG. 1. The processor 302 used in the operator console 120 is an example of a “first processor” according to the present disclosure.

### Overview of Algorithm for Determining State of Slip Ring 111

The processor 302 used in the state determination apparatus 140 can function as the image computation device 122, the system control device 124, and the determination unit 126.

The image computation device 122 recognizes the image acquired from the camera 113 and detects a region of an object such as the power slip ring 111A, the control slip ring 111B, the groove 154, and the deposits in the image. As an image recognition technology of detecting the region of the object from the image, for example, an algorithm of machine learning such as deep learning or an algorithm of pattern matching can be applied. The image computation device 122 includes, for example, a trained model that has been trained to perform a task of region classification (semantic segmentation), and may detect the region of the object from the image using the trained model. The trained model is constructed using, for example, a convolutional neural network. The “model” is, in essence, a program.

The image computation device 122 calculates the feature value from the image based on the recognition result of the image, and analyzes the amount of change in the feature value. The image computation device 122 may calculate the feature value for each region of the recognized object. The feature value may be, for example, a value indicating luminance (brightness) calculated from a signal value (pixel value) of each component of RGB of the image, a distribution of the brightness, a chromaticity value indicating a color, a distribution of the color, a distribution obtained by edge (brightness difference) detection, and the like. The feature value may be a feature value calculated by using a convolutional neural network or the like. The image computation device 122 calculates one or more feature values, preferably a plurality of types of feature values, from the image.

The determination unit 126 quantitatively determines the state of the object including the state of the sliding surface of the slip ring 111 using the threshold value, based on the feature value calculated by the image computation device 122. The determination unit 126 can determine whether the maintenance is necessary based on the feature value calculated from the image and the set threshold value. In the state determination apparatus 140, the image and the determination result are displayed on the display device 125 such that the surface state of the slip ring 111 can be visually checked by the image.

The state of the object that is evaluated based on the feature value obtained from the image may include at least one of a degree of contamination of the slip rings 111A and 111B, a degree of contamination of the groove 154, an amount of deposits adhering to the slip rings 111A and 111B, or an amount of deposits adhering to the groove 154.

The image computation device 122 may calculate the feature value differently depending on the type of the object. For example, the feature value measured for the region of the slip rings 111A and 111B and the feature value measured for the groove 154 may be different types of feature values. In addition, in the determination unit 126, the threshold value may be set differently depending on the type of the object. For example, the threshold value of the abnormality determination may be different between the power slip ring 111A and the control slip ring 111B.

The threshold value of the abnormality determination is optimized by statistical processing, and the determination is performed without depending on the state when the feature value is measured. The term “optimization” is not limited to strict optimization, and includes the concept of approaching the optimum. The phrase “when the feature value is measured” may be understood as when the image for calculating the feature value is captured (when the image is acquired). The term “when” in the phrase “when . . . is measured” is not limited to a point in time, and includes a time range that is reasonably allowable.

### Second Embodiment: Example of System that Changes Acquisition Frequency of Image by Camera 113

In a second embodiment, in addition to the configuration of the first embodiment, in order to prevent over-or under-diagnosis of the slip ring 111 by the state determination apparatus 140, a condition for changing the acquisition frequency of the image is set in advance, and the acquisition frequency of the image is changed in a case in which the analysis result of the acquired image satisfies the condition. The condition for changing the acquisition frequency of the image will be referred to as a “frequency change condition”. The acquisition frequency of the image is synonymous with the measurement frequency of the feature value.

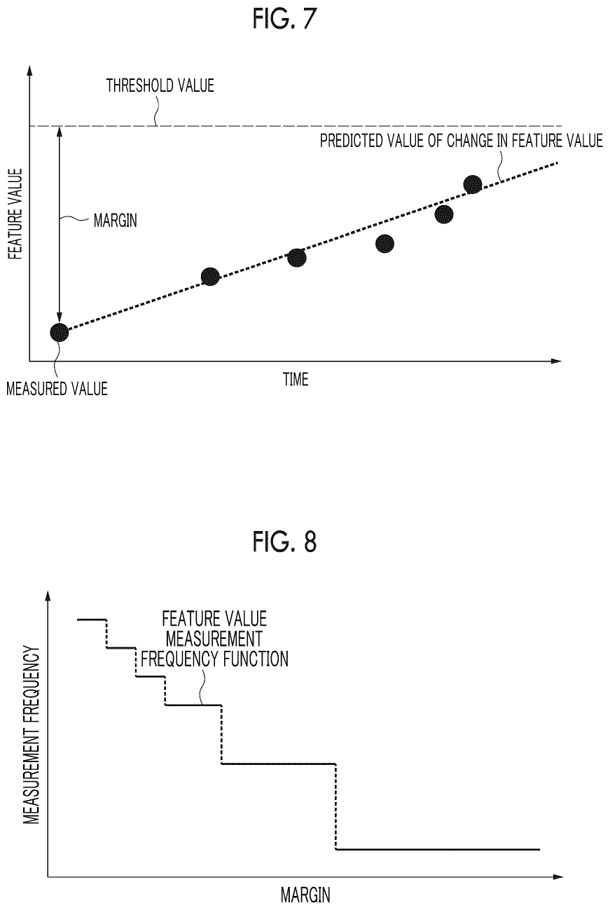

An example of the system that changes the measurement frequency of the feature value will be described with reference to FIGS. 7 to 9. The frequency change condition may be determined based on, for example, a difference between the measured value of a certain feature value and the threshold value of the abnormality determination. The difference between the measured value of the feature value and the threshold value of the abnormality determination will be referred to as “margin magnitude”.

FIG. 7 is a graph illustrating a transition of the measured value of a certain feature value. A horizontal axis indicates time, a vertical axis indicates the feature value calculated (measured) from the image, and a black circle is a point at which the measured value of the feature value is plotted. The feature value illustrated in FIG. 7 tends to increase as the degree of contamination of the slip ring 111 increases. As illustrated in FIG. 7, a predicted value of the change in the feature value can be obtained from the results of a plurality of measured values.

FIG. 8 is a graph illustrating an example of the frequency change condition set in advance. A horizontal axis indicates the margin magnitude, and a vertical axis indicates the measurement frequency. As illustrated in FIG. 8, a feature value measurement frequency function is set such that the measurement frequency is set to be small in a region in which the margin is large, and the measurement frequency is gradually increased as the margin decreases. The feature value measurement frequency function will be referred to as a “measurement frequency function”.

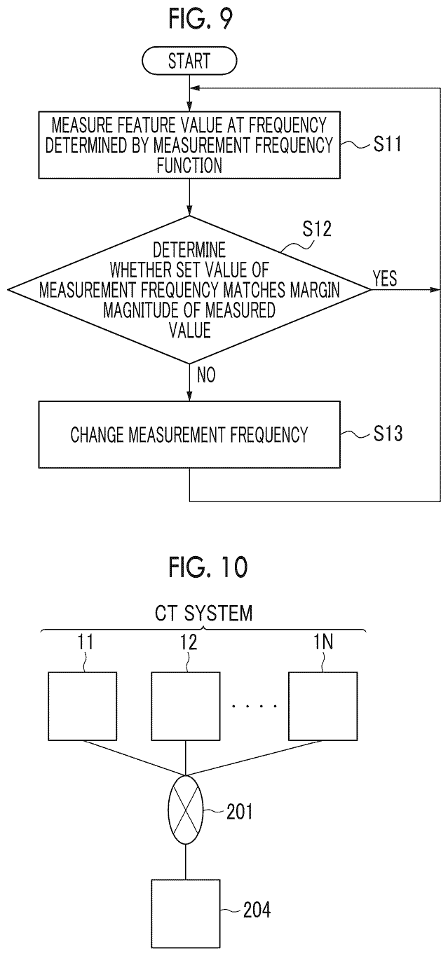

FIG. 9 is a flowchart illustrating an example of a process of changing the measurement frequency of the feature value. In step S11, the processor 302 executes the measurement of the feature value at a frequency defined by the measurement frequency function set as illustrated in FIG. 8. In an initial state, since the margin is sufficiently large, a minimum value of the measurement frequency defined by the measurement frequency function may be applied as an initial set value of the measurement frequency. The initial set value may be, for example, once per month (frequency of once a month).

In step S12, the processor 302 determines whether the set value of the measurement frequency matches the margin magnitude of the measured value. In a case in which the determination result in step S12 is YES, the process returns to step S11.

In a case in which the determination result in step S12 is NO, the process proceeds to step S13. In step S13, the processor 302 changes the measurement frequency to the measurement frequency corresponding to the margin magnitude of the measured value from the measurement frequency function. After step S13, the process returns to step S11.

As described above, it is desirable that the measurement frequency of the feature value is changed depending on the margin magnitude of the measured value of the feature value with respect to the threshold value of the abnormality determination. As the margin magnitude decreases, it is desirable to increase the measurement frequency.

As a result, in a case in which the margin magnitude of the measured value of a certain feature value is equal to or less than a certain value, the process of changing the measurement frequency from, for example, once per month to once per week (frequency of once a week) is performed.

The processor 302 of the state determination apparatus 140 according to the second embodiment functions as a frequency change condition setting unit that sets the frequency change condition, and functions as a measurement frequency determination unit that determines the measurement frequency of the feature value based on the set frequency change condition and the feature value calculated from the image.

### Third Embodiment

### Specific Example 1 of Method of Optimizing Measurement Condition of Feature Value: Changing Threshold Value

In a third embodiment, an example of a method of changing the threshold value of the abnormality determination to an appropriate value will be described. FIG. 10 is a diagram illustrating an example of a configuration in which a plurality of CT systems are connected to the maintenance management apparatus 202 via the network 201.

The maintenance management apparatus 202 collects various measured values of the feature values of each of a plurality of CT systems 11, 12, . . . , 1N and the status such as whether a system outage has occurred from the plurality of CT systems 11, 12, . . . , 1N via the network 201. Each of the CT systems 11, 12, . . . , 1N may have the same configuration as the X-ray CT apparatus 1 illustrated in FIG. 1.

Any status is transmitted from each of the CT systems 11, 12, . . . , 1N connected to the network 201 to the maintenance management apparatus 202 located externally. The CT systems 11, 12, . . . , 1N can transmit at least one of the feature value calculated from the image, the status of the system, or the operation information of the system to the maintenance management apparatus 202.

The status transmitted from the CT systems 11, 12, . . . , 1N to the maintenance management apparatus 202 may include, for example, a margin of the measured value of the feature value, the number of revolutions of the slip ring 111, a communication failure count, and a cleaning date. Here, the communication failure count is the number of communication failures caused by the contamination of the control slip ring 111B. The cleaning date is a date on which the slip ring 111 is cleaned.

The maintenance management apparatus 202 performs optimization or classification of various conditions by statistical processing from the status collected from each system. For the optimization of various conditions, for example, a machine learning model such as a support vector machine (SVM) may be applied. In addition, for the classification of various conditions, for example, a clustering algorithm using a k-means method (k-means clustering) may be applied. The maintenance management apparatus 202 transmits the optimized various conditions to each system via the network 201 to change various determination conditions.

For example, in a case in which the desired classification cannot be performed with the threshold value set in advance, the maintenance management apparatus 202 uses the data for which the desired classification cannot be performed to perform, for example, supervised learning such as SVM, and sets a new threshold value.

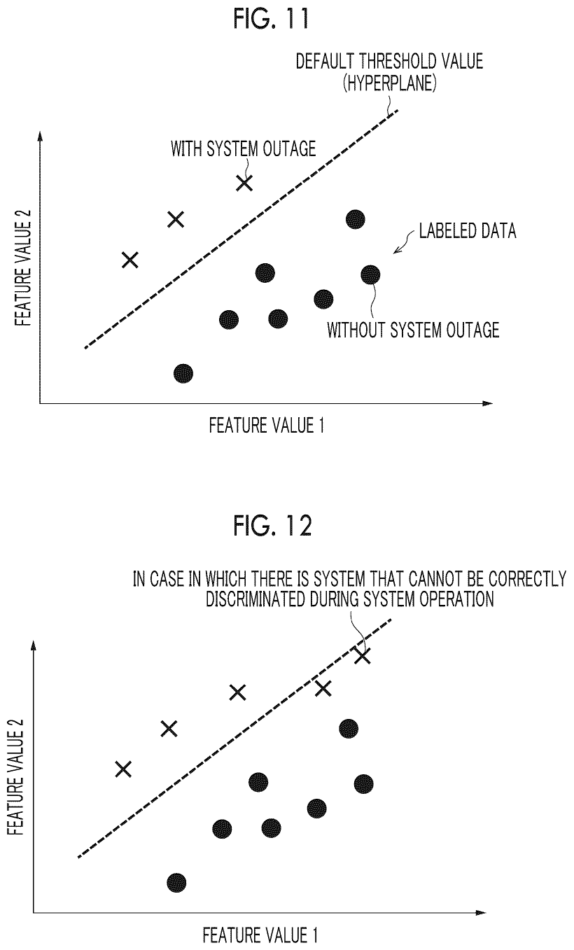

FIG. 11 is a graph illustrating an example of a default threshold value setting. In FIG. 11, for convenience of illustration, a two-dimensional feature value space of a feature value 1 and a feature value 2 is used for the description, but an actual feature value space may be any multi-dimensional space. A black circle in FIG. 11 represents data without the system outage, and an “×” mark represents data with the system outage. The “system outage” includes a state in which the use of the CT system is impossible due to the contamination of the slip ring 111 and the like. Each of a plurality of data illustrated in FIG. 11 is labeled data, and a set of the plurality of data is a training dataset of the SVM.

As illustrated in FIG. 11, from the training dataset, a hyperplane (separating hyperplane) that is a boundary for classifying a data group with the system outage and a data group without the system outage is determined, and the hyperplane can be used as the default threshold value.

Thereafter, in a case in which the desired classification cannot be performed with the default threshold value, the data for which the desired classification cannot be performed is used to perform, for example, supervised learning such as SVM, and a new threshold value is set.

The example thereof is illustrated in FIG. 12. FIG. 12 illustrates that data of the system for which correct discrimination cannot be performed during system operation is added to the initial training dataset illustrated in FIG. 11. The added data is classified as “without system outage” in a case in which the default threshold value is applied, but, in the actual system, indicates a case in which the system outage has occurred.

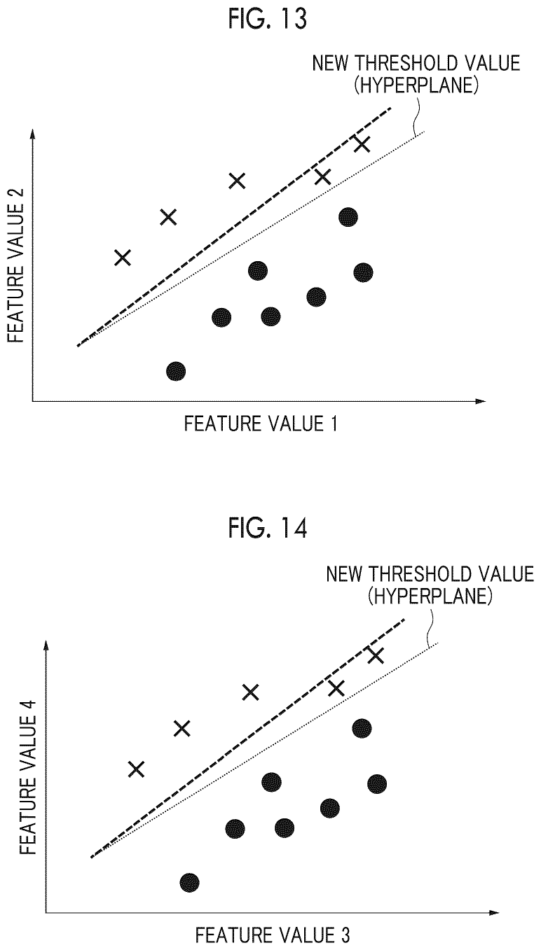

In such a case, as illustrated in FIG. 13, the maintenance management apparatus 202 performs supervised learning using a dataset including data that cannot be appropriately classified, obtains a new hyperplane that can appropriately classify the data, and determines a new threshold value.

Alternatively, as illustrated in FIG. 14, the maintenance management apparatus 202 sets the threshold value at which the desired classification result is obtained, by changing the feature value used for the classification.

FIG. 14 illustrates an example in which a combination of the feature value 1 and the feature value 2 in FIG. 12 is changed to a combination of a feature value 3 and a feature value 4. For convenience of illustration, the data plot points are not changed between FIG. 12 and FIG. 14, but in practice, since the combination of the feature values (feature value space) is different, the data plot points in FIG. 14 may be different from those in FIG. 12.

The maintenance management apparatus 202 provides the new threshold value or the feature value obtained in this way to each CT system via the network 201, and changes the threshold value or the feature value used in each system.

The hardware configuration of the maintenance management apparatus 202 may be the same as the hardware configuration of the information processing apparatus 300 illustrated in FIG. 6. The processor used in the maintenance management apparatus 202 is an example of a “second processor” according to the present disclosure.

The processor of the maintenance management apparatus 202 according to the third embodiment functions as a setting information determination unit that optimizes various conditions including the determination conditions such as the threshold value based on the information obtained from the plurality of CT systems 11, 12, . . . , 1N, and determines the setting information applied to the determination, and functions as a setting information providing unit that provides the determined setting information to each system via the network 201.

### Fourth Embodiment

### Specific Example 2 of Method of Optimizing Measurement Condition of Feature Value: Changing Measurement Frequency

In a fourth embodiment, an example of a method of changing the measurement frequency function to an appropriate function will be described. The maintenance management apparatus 202 collects information on the number of revolutions and the rotational speed of the slip ring 111 of each system via the network 201. Then, the maintenance management apparatus 202 classifies the systems by using, for example, a k-means clustering method.

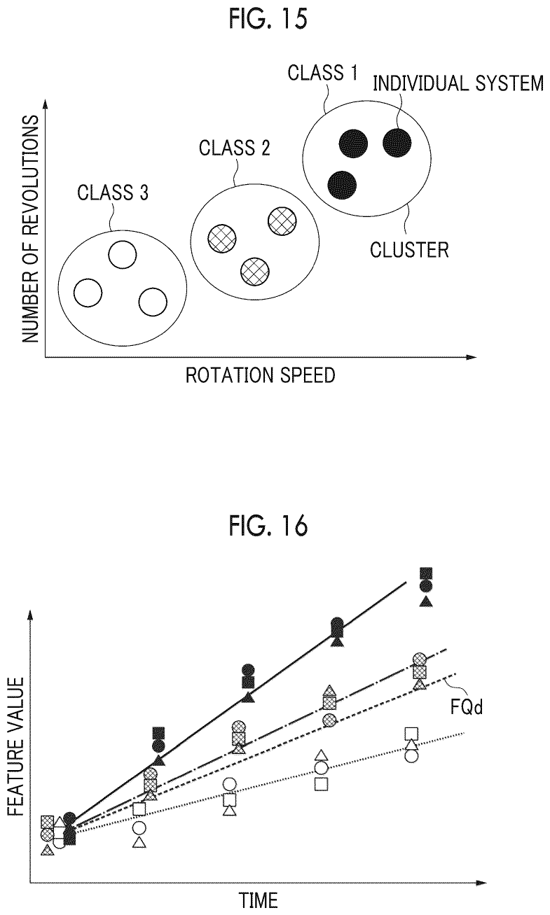

FIG. 15 is a conceptual diagram of the classification. FIG. 15 illustrates an example of data of nine systems, and these plurality of systems are classified into three classes (clusters) of “class 1”, “class 2”, and “class 3”. The maintenance management apparatus 202 calculates an average of the amounts of change in the feature values for each class based on the classification.

FIG. 16 is a graph illustrating the average of the amounts of change in the feature values for each class. A graph FQd illustrated by a broken line in FIG. 16 is an estimated value of the amount of change in the feature value estimated by default. A graph illustrated by a solid line in FIG. 16 illustrates an average value of the amounts of change in the feature values of the systems belonging to the class 1, a graph illustrated by a dashed-dotted line illustrates an average value of the amounts of change in the feature values of the systems belonging to the class 2, and a graph illustrated by a dotted line illustrates an average value of the amounts of change in the feature values of the systems belonging to the class 3.

The maintenance management apparatus 202 compares the estimated value (graph FQd) of the amount of change in the feature value estimated in the first embodiment or the like with the average value of the amounts of change in the feature values calculated from each class, and changes the measurement frequency function for each class. The average value is an example of a “representative value” according to the present disclosure.

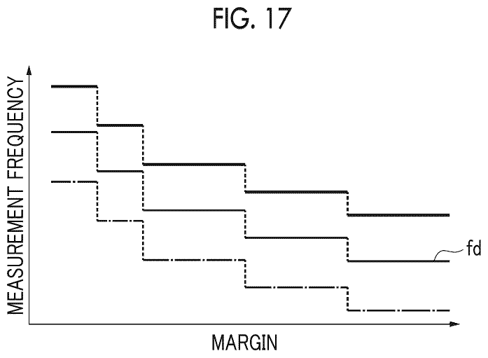

FIG. 17 is an example of the measurement frequency function. A measurement frequency function fd illustrated by a solid line in FIG. 17 is a default measurement frequency function determined from the estimated value of the change of the feature value estimated by default. Each of the measurement frequency function illustrated by a thick solid line and the frequency function illustrated by a dashed-dotted line in FIG. 17 is the measurement frequency function calculated for each class illustrated in FIG. 16.

In this manner, the set value of the measurement frequency function calculated for each class is fed back to each system of the corresponding class via the network 201. The information on the measurement frequency function provided from the maintenance management apparatus 202 to each system is an example of “condition setting information” according to the present disclosure. The processor 302 on the CT system side acquires information on the measurement frequency function from the maintenance management apparatus 202 via the network 201, and changes (updates) the measurement frequency function to be applied.

The processor of the maintenance management apparatus 202 according to the fourth embodiment functions as a classification unit that classifies the systems based on the information obtained from the plurality of CT systems 11, 12, . . . , 1N, and functions as a measurement frequency condition determination unit that determines an appropriate measurement frequency function for each class of the classified systems.

### About Processor

In the embodiments of the present disclosure, each processing is executed by any computer. In addition, any computer may execute the processing by a processor, a program, or a combination thereof. Any computer may be a general-purpose computer, a computer for a specific purpose, a system such as a workstation, or other hardware components capable of executing a program.

The processor may be implemented by one or more hardware components, and the type of hardware is not limited. The processor may be configured by, for example, a central processing unit (CPU), a micro processing unit (MPU), a programmable logic device such as a field programmable gate array (FPGA), a dedicated circuit for executing specific processing, such as an application specific integrated circuit (ASIC), or hardware such as a graphics processing unit (GPU) or a neural processing unit (NPU).

Further, the processor includes each unit or each means that executes various types of processing in the present embodiment. In addition, the type of hardware may be a combination of different kinds of hardware. In a case in which the plurality of types of hardware are configured to execute one or a plurality of processes of a certain processor, the plurality of types of hardware may be present in devices physically separated from each other or may be present in the same device. Further, in any of the embodiments, the order of each process performed by the processor is not limited to the above-described order, and may be changed as appropriate. The hardware is configured by an electric circuit (circuitry) in which circuit elements, such as semiconductor elements, are combined, or the like.

Further, the present embodiment may be implemented by hardware, software, firmware, microcode, or a combination thereof. Software, firmware, and microcode are configured by a program. The program may be, for example, a group of program modules, and each function thereof may be implemented by a processor configured to execute each function. The program may be a program code or a plurality of code segments stored in one or more non-transitory computer-readable media (for example, a storage medium and other storages). The program may be stored in the plurality of non-transitory computer-readable media present in devices physically separated from each other. The program code or the code segment may represent any combination of procedures, functions, subprograms, routines, subroutines, modules, software packages, classes, or commands, data structures, or program statements. The program code or the code segment may be connected to another code segment or a hardware circuit by transmitting and receiving information, data, an argument, a parameter, or a content of a memory.

### About Program and Program Product for Operating Computer

The method of processing in the embodiments of the present disclosure may be configured as a program or a program product for causing a processor or a computer including the processor to implement functions of each step. The program product is a computer-readable medium that is a tangible, non-transitory information storage medium on which a program is recorded.

It is possible to record a program causing a computer to realize some or all of the processing functions of the state determination apparatus 140 and the maintenance management apparatus 202, on an optical disk, a magnetic disk, or a computer-readable medium such as a semiconductor memory or other tangible non-transitory information storage medium, and to provide the program through this information storage medium.

Alternatively to providing the program stored on such a tangible, non-transitory computer-readable medium, the program signal may be provided as a download service via a communication network such as the Internet.

Further, some or all of the processing functions of the state determination apparatus 140 and the maintenance management apparatus 202 may be implemented by cloud computing, and may also be provided as software as a service (SaaS).

### Advantages of Embodiment

The embodiments of the present disclosure have the following advantages.
[1] With the state determination apparatus 140, it is possible to quantitatively determine the degree of contamination of the slip ring 111, the degree of contamination of the groove 154 between the rings, the amount of deposits such as carbon shaving chips adhering to the groove 154, and the like based on the feature value calculated from the image of the region including the slip ring 111.
[2] With the state determination apparatus 140, the image of the region including the slip ring 111 can be displayed on the display device 125, and the surface state of the slip ring 111 can be visually checked.
[3] In a case in which the determination unit 126 determines that the state requiring the maintenance is present, the warning can be issued through the screen of the display device 125.
[4] With the state determination apparatus 140, it is possible to identify each region of the power slip ring 111A, the control slip ring 111B, and the groove 154 between the rings from the image obtained from the camera 113, and determine the degree of contamination of each ring, the degree of contamination of the groove 154, the amount of deposits, and the like, and it is possible to predict the maintenance timing based on the determination result.
[5] With the X-ray CT apparatus 1 in which the state determination apparatus 140 is incorporated, it is possible to self-diagnose the surface state of the slip ring 111.
[6] With the state determination apparatus 140 according to the second embodiment, the image can be acquired at an appropriate frequency in accordance with the margin magnitude of the measured value of the feature value with respect to the threshold value of the abnormality determination, and the state of the slip ring 111 can be diagnosed. According to the second embodiment, the state of the slip ring 111 can be diagnosed at an appropriate timing without over-or under-diagnosis.
[7] With the maintenance management apparatus 202 according to the third embodiment, the determination conditions in each CT system can be optimized, and the determination accuracy can be improved.
[8] With the maintenance management apparatus 202 according to the fourth embodiment, an appropriate measurement frequency can be set for each CT system, and the optimization of the diagnosis timing can be achieved.

### MODIFICATION EXAMPLE 1

In FIG. 6, an example has been described in which two types of slip rings, which are the power slip ring 111A and the control slip ring 111B, are provided, but one type of the slip ring may be used. In addition, in the configuration in which two or more types of slip rings are provided, the slip rings to be diagnosed may be some of the slip rings.

### MODIFICATION EXAMPLE 2

The technology of the present disclosure can be applied to various apparatuses comprising a slip ring, not limited to the medical imaging apparatus that is substituted for the X-ray CT apparatus 1.

### OTHERS

The configurations described in each of the embodiments described above and the features described in the modification examples may be used in appropriate combinations, and some features may be substituted. The present disclosure is not limited to the embodiments described above, and various modifications can be made without departing from the gist of the technical idea of the present disclosure.

### EXPLANATION OF REFERENCES

1: X-ray CT apparatus
11, 12, 1N: CT system
100: scan gantry unit
101: X-ray tube
102: rotating disk
103: collimator
104: opening part
105: table
106: X-ray detector
107: data collection device
108: gantry control device
109: table control device
110: X-ray control device
111: slip ring
111A: power slip ring
111B: control slip ring
112, 112A, 112B: lighting device
113: camera
114: light-shielding guard
116: half mirror
120: operator console
121: input device
122: image computation device
123: storage device
124: system control device
125: display device
126: determination unit
140: state determination apparatus
150: power slip ring unit
152: control slip ring unit
154: groove
201: network
202: maintenance management apparatus
300: information processing apparatus
302: processor
304: memory
306: storage
308: input/output interface
310: bus
312: input device
314: display device
FQd: graph

fd: measurement frequency function
IM: image
S11 to S13: steps of method of changing measurement frequency of feature value

What is claimed is:

1. A state determination apparatus comprising:
a lighting device that irradiates a slip ring with light;
a camera that captures an image of a region including the slip ring; and
a processor that recognizes the image acquired via the camera and determines a state of an object based on a recognition result of the image.

2. The state determination apparatus according to claim 1,
wherein the state of the object includes at least one of a degree of contamination of the slip ring, a degree of contamination of an inter-ring region of the slip ring, or an amount of deposits in the inter-ring region.

3. The state determination apparatus according to claim 1,
wherein the processor
calculates a feature value from the image, and
determines whether maintenance is necessary based on the feature value and a threshold value that is set.

4. The state determination apparatus according to claim 1,
wherein the image includes at least one of
a power slip ring for power transmission, or
a control slip ring for signal transmission.

5. The state determination apparatus according to claim 1,
wherein the image includes a groove of an inter-ring region of the slip ring.

6. The state determination apparatus according to claim 1,
wherein the image is captured using diffused light.

7. The state determination apparatus according to claim 6,
wherein the lighting device irradiates the region including the slip ring with light from an oblique direction.

8. The state determination apparatus according to claim 6,
wherein a plurality of the lighting devices that irradiate the region including the slip ring with light from a plurality of directions are provided.

9. The state determination apparatus according to claim 6, further comprising:
a light-shielding guard that suppresses direct incidence of light from the lighting device to the camera.

10. The state determination apparatus according to claim 1, wherein the image is captured using specularly reflected light.

11. The state determination apparatus according to claim 10, further comprising:
a half mirror that is disposed between the lighting device and the slip ring and reflects the specularly reflected light from the slip ring toward the camera.

12. The state determination apparatus according to claim 1,
wherein the processor determines an acquisition frequency of the image based on a feature value calculated from the image.

13. The state determination apparatus according to claim 12,
wherein the processor
sets a condition for changing the acquisition frequency of the image, and
changes the acquisition frequency of the image in a case in which the feature value satisfies the condition.

14. The state determination apparatus according to claim 12,
wherein the processor changes the acquisition frequency of the image in accordance with a difference between the feature value and a threshold value that is set.

15. A maintenance management apparatus connected to a system including the state determination apparatus according to claim 1 via a network, the maintenance management apparatus comprising:
a second processor different from a first processor that is the processor included in each of a plurality of the systems,
wherein the second processor
acquires information from the plurality of systems via the network,
determines setting information applied to the determination in the system based on the information acquired from the plurality of systems, and
provides the setting information to the systems via the network.

16. The maintenance management apparatus according to claim 15,
wherein the information acquired by the second processor from the plurality of systems includes at least one of a feature value calculated from the image of the system, a status of the system, or operation information of the system, and
the setting information includes at least one of a threshold value used for the determination, a type of the feature value used for the determination, an acquisition frequency of the image, or a condition for changing the acquisition frequency of the image.

17. The maintenance management apparatus according to claim 16,
wherein the second processor
acquires the feature value and the status indicating whether a system outage has occurred from the plurality of systems, and
determines at least one of the threshold value used for the determination or the type of the feature value used for the determination based on a dataset including the acquired feature value and the acquired status.

18. The maintenance management apparatus according to claim 16,
wherein the operation information of the system includes information on the number of revolutions and a rotational speed of a rotating member provided with the slip ring in the system, and
the second processor
acquires the information on the number of revolutions and the rotational speed of each system and the feature value from the plurality of systems,
classifies the plurality of systems using the information on the number of revolutions and the rotational speed,
calculates a representative value of an amount of change in the feature value for each class of the classified systems, and
determines the condition for changing the acquisition frequency of the image for each class based on a comparison result between the representative value and an estimated value of the amount of change as a reference.

19. An X-ray CT apparatus comprising:
an X-ray source that irradiates a subject with X-rays;
an X-ray detector that is disposed to face the X-ray source and detects the X-rays transmitted through the subject;
a rotating member that is provided with the X-ray source and the X-ray detector and rotates around the subject;
a slip ring that is provided on the rotating member;
a lighting device that irradiates the slip ring with light;
a camera that captures an image of a region including the slip ring; and
a processor that recognizes the image acquired via the camera and determines a state of an object based on a recognition result of the image.

20. An information processing apparatus comprising:
a processor,
wherein the processor
acquires an image of a region including a slip ring,
recognizes the image, and
determines a state of an object based on a recognition result of the image.
